(19) United States
(12) Patent Application Publication
(10) Pub. No.: US 2009/0326390 A1
(43) Pub. Date: Dec. 31, 2009

(54) Pressure and Oxygen Saturation Monitoring Devices and Systems

(76) Inventors: Andres Belalcazar, St. Paul, MN (US); Justin R. Van Hee, Minneapolis, MN (US); Lynn M. Zwiers, Lino Lakes, MN (US); Jennifer L. Pavlovic, Afton, MN (US)

Correspondence Address: FISH & RICHARDSON P.C., P.O. BOX 1022, MINNEAPOLIS, MN, 55440-1022, US

(21) Appl. No.: 12/164,870

(22) Filed: Jun. 30, 2008

(51) Int. Cl. A61B 5/00

(52) U.S. Cl. 600/486; 600/327

(57) ABSTRACT

Pressure and oxygen saturation monitoring devices and systems are disclosed. The devices, or portions thereof, can be implanted within a subject for monitoring blood pressure and oxygen saturation.

### FIELD OF THE INVENTION

The present invention relates to pressure and oxygen saturation monitoring devices and systems.

### BACKGROUND

Blood pressure measurements are important for medical research and clinical diagnosis. Such measurements provide researchers and clinicians with insight into the physiology and functioning of the cardiovascular system. Oxygen saturation is also an important parameter used to evaluate cardiovascular and respiratory function in clinical and research environments. Blood pressure and oxygen saturation measurements can provide useful information regarding the safety and efficacy of pharmaceuticals and toxic chemicals.

### SUMMARY

Pressure and oxygen saturation monitoring devices and systems are disclosed. In one aspect, an implantable device for monitoring blood pressure and oxygen saturation in a living being includes a fluid filled catheter having a distal portion adapted for insertion within a blood vessel lumen of the living being. The device further includes a pressure sensor in communication with the fluid, the fluid communicating blood pressure to the pressure sensor for transduction of the blood pressure into electric signals. In addition to the pressure sensor and catheter, the device includes a light source adapted for illuminating blood within the blood vessel lumen and a photodetector adapted to sample light reflected by the blood located in the blood vessel lumen. The photodetector transduces the sampled light into electrical signals.

Various embodiments of the implantable device may include one or more of the following features. The device may include signal processing circuitry electrically coupled to the pressure sensor and photodetector. The signal processing circuitry may be adapted for determining blood pressure and oxygen saturation measurements based upon the electrical signals transduced from the blood pressure and the electrical signals transduced from the sampled light. The signal processing circuitry may be adapted for remotely transmitting data for determining blood pressure and oxygen saturation based upon the electrical signals transduced from the blood pressure and the electrical signals transduced from the sampled light. The light source may be positioned in relation to the catheter to project light into the fluid of the catheter for illuminating the blood within the vessel. The fluid may be configured to transmit the light projected into it. The light projected into the fluid may be directed into the blood vessel lumen by transmission through the fluid. In the described device, the photodetector may be positioned in relation to the catheter to sample the light reflected by blood located in the vessel lumen. The light reflected by the blood located in the vessel lumen may be guided to the photodetector for sampling by transmission into and through the fluid.

The described implantable device may further include an optical fiber. The light source may be positioned to project light into the optical fiber. In such a case, the optical fiber may be adapted to transmit the light received from the light source to illuminate blood within the blood vessel. The optical fiber may also be adapted to receive light reflected by blood located within the blood vessel lumen and to transmit the received reflected light to the photodetector for sampling. The light source may be positioned to project light into the optical fiber and the photodetector may be positioned to sample light reflected by blood within the blood vessel lumen and transmitted to the photodetector through the fluid. In some aspects, the light source may be positioned to project light into the fluid for illuminating blood within the blood vessel lumen and the photodetector may be positioned to sample light reflected by the blood and transmitted to the photodetector through the optical fiber. The device may further include a second optical fiber. The second optical fiber may be adapted to receive light reflected by blood within the blood vessel lumen and to transmit the received reflected light to the photodetector for sampling.

In some aspects, the fluid filled catheter may have at least a first and second lumen, with each lumen being fluid filled. When the fluid filled catheter has at least two fluid filled lumens, the light source may be positioned to project light into the first fluid filled lumen for illuminating blood with the blood vessel lumen and the photodetector may be positioned to sample light reflected by blood within the blood vessel lumen and transmitted to the photodetector through the fluid of the second lumen.

In one aspect, a system for monitoring blood pressure and oxygen saturation in a living being includes an implantable device, the implantable device including a fluid filled catheter having a distal portion adapted for insertion within a blood vessel lumen of the living being. The implantable device of the system further includes a pressure sensor in communication with the fluid, the fluid communicating blood pressure to the pressure sensor for transduction of the blood pressure into electric signals, a light source adapted for illuminating blood within the blood vessel lumen, and a photodetector adapted to sample light reflected by the blood located in the blood vessel lumen and that transduces the sampled light into electrical signals. The described system further includes signal processing circuitry electrically coupled to the pressure sensor and photodetector. The signal processing circuitry is adapted for remotely transmitting data for determining blood pressure and oxygen saturation based upon the electrical signals transduced from the blood pressure and the electrical signals transduced from the sampled light. The system further includes a remote processing unit located external to the living being and configured to receive the transmitted data and to determine blood pressure and oxygen saturation values from the received transmitted data.

Various embodiments of the system may include one or more of the following features. The light source may be positioned in relation to the catheter to project light into the fluid of the catheter for illuminating the blood within the vessel. In this aspect, the fluid may be configured to transmit the light projected into it. Light projected into the fluid may be directed into the blood vessel lumen by transmission through the fluid.

The photodetector of the system may be positioned in relation to the catheter to sample the light reflected by blood located in the vessel lumen. In this aspect, light reflected by the blood located in the blood vessel lumen may be guided to the photodetector for sampling by transmission into and through the fluid.

The system may further include an optical fiber. The light source may be positioned to project light into the optical fiber and the optical fiber may be adapted to transmit the light received from the light source to illuminate blood within the blood vessel. The optical fiber may also be adapted to receive light reflected by blood located within the blood vessel lumen and to transmit the received reflected light to the photodetector for sampling. In this aspect, the photodetector may be positioned in relation to the optical fiber to sample the reflected light transmitted to it by the optical fiber. The light source may be positioned to project light into the fluid for illuminating blood within the blood vessel lumen. The photodetector may be positioned to sample light reflected by the blood and transmitted to the photodetector through the optical fiber. In some aspects, the light source may be positioned to project light into the optical fiber and the photodetector may be positioned to sample light reflected by blood within the blood vessel lumen and transmitted to the photodetector through the fluid. The system may further include a second optical fiber adapted to receive light reflected by blood within the blood vessel lumen and to transmit the received reflected light to the photodetector for sampling.

In some aspects, the catheter of the system may have at least a first and second lumen, with each lumen being fluid filled. In these aspects, the light source may be positioned to project light into the first fluid filled lumen for illuminating blood with the blood vessel lumen and the photodetector may be positioned to sample light reflected by blood within the blood vessel lumen and transmitted to the photodetector through the fluid of the second lumen.

Another embodiment of an implantable device for monitoring blood pressure and oxygen saturation in a living being includes a fluid filled catheter having a distal portion adapted for insertion within a blood vessel lumen of the living being. A membrane is positioned in the distal portion of the catheter and coupled to the fluid. The membrane is adapted to be deflected in response to blood pressure in the blood vessel lumen. The device further includes a light source adapted for illuminating blood within the blood vessel lumen and a photodetector adapted to sample light reflected by the blood located in the blood vessel lumen and to sample light modulated by deflection of the membrane. The photodetector transduces the sampled light into electrical signals. In one aspect, the device may further include signal processing circuitry electrically coupled to the photodetector for determining blood pressure and oxygen saturation measurements based upon the electrical signals transduced from the sampled light. In one aspect, the device may further include signal processing circuitry electrically coupled to the photodetector for remotely transmitting data for determining blood pressure and oxygen saturation based upon the electrical signals transduced from the sampled light.

The devices, systems, or portions thereof, can be implanted within a subject, including a human or non-human animal, for monitoring blood pressure and oxygen saturation. For example, in research animals, researchers have observed that when an animal's blood is sampled the oxygen saturation reading is generally very high and does not vary, which can indicate that a disturbed and excited animal may mask a low oxygen saturation reading. In another example, in poorly ventilated humans, excitement or talking can mask a low oxygen saturation. In both examples, because blood saturates quickly, oxygen saturation readings from blood samples can be artificially elevated due to excitement or sampling procedures. Blood pressure can also be elevated by excitement or sampling procedures. The described devices and systems may be used to monitor blood pressure and oxygen saturation and can reduce unwanted elevated oxygen saturation readings. The devices may be used to monitor research animals to provide oxygen saturation readings less affected by temporary excitement induced by handling, while also providing accurate blood pressure readings. The devices may also be used clinically in human or veterinary patients. For example, patients where oxygen saturation is being chronically measured, ventilated patients, or those with heart failure or receiving oxygen therapy may be advantageously monitored using the described devices.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic diagram illustrating an implantable device for sensing blood pressure and oxygen saturation.

FIG. 2 is a schematic cross-sectional diagram illustrating aspects of an example embodiment of a device for sensing blood pressure and oxygen saturation.

FIG. 3 is a schematic cross-sectional diagram illustrating aspects of an example embodiment of a device for sensing blood pressure and oxygen saturation.

FIG. 4 is a schematic cross-sectional diagram illustrating a cross section taken across line 4-4 of FIG. 2 or FIG. 3.

FIG. 5 is a schematic cross-sectional diagram illustrating aspects of an example embodiment of a device for sensing blood pressure and oxygen saturation.

FIG. 6 is a schematic cross-sectional diagram further illustrating aspects of the pressure and light sensing catheter of FIG. 2.

FIG. 7 is a schematic cross-sectional diagram further illustrating aspects of an example pressure sensing unit and an optical unit.

FIG. 8 is a schematic cross-sectional diagram illustrating aspects of an example embodiment of a device for sensing blood pressure and oxygen saturation having a optical fiber.

FIG. 9 is a schematic cross-sectional diagram further illustrating aspects of the pressure and light sensing catheter of FIG. 8.

FIG. 10A is a schematic cross-sectional diagram illustrating a cross section taken across line 10A-10A of FIG. 8.

FIG. 10B is a schematic cross-sectional diagram illustrating an embodiment of a pressure and light transmission catheter comprising an optical fiber.

FIG. 11 is a schematic cross-sectional diagram illustrating aspects of an example embodiment of a device for sensing blood pressure and oxygen saturation having a pressure and light transmission catheter comprising two lumens.

FIG. 12A is a schematic cross-sectional diagram illustrating aspects of an example embodiment of a device for sensing blood pressure and oxygen saturation having a pressure and light transmission catheter comprising an optical fiber.

FIG. 12B is a schematic cross-sectional diagram illustrating aspects of an example embodiment of a device for sensing blood pressure and oxygen saturation having a pressure and light transmission catheter comprising an optical fiber.

FIG. 13 is a schematic cross-sectional diagram illustrating aspects of an example embodiment of a device for sensing blood pressure and oxygen saturation.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of what is claimed.

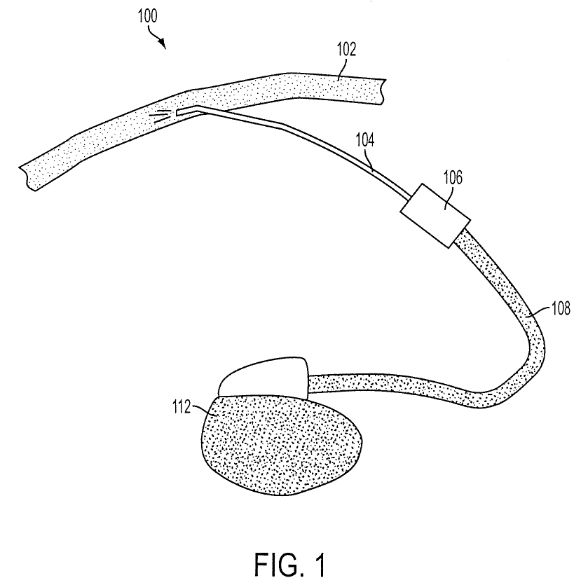

FIG. 1 is a schematic diagram of an example device 100 configured to measure and/or monitor blood pressure and oxygen saturation in a subject. The device comprises a pressure and light transmission catheter 104. A distal portion of the pressure and light transmission catheter 104 can be positioned within a subject or living being for measuring and/or monitoring blood pressure and oxygen saturation. For example, a distal portion of the catheter 104 can be positioned within a blood vessel 102 of a subject. The blood vessel can be an artery, vein, cardiac chamber or any other location within a subject or living being where blood pressure and oxygen saturation is desirably monitored. The catheter can be positioned within the lumen of any of these blood vessel structures to monitor blood pressure and oxygen saturation.

The catheter 104 can be configured to transmit pressure and light to a sensor housing 106. The sensor housing 106 comprises a pressure sensing unit and an optical unit. The pressure sensing unit can generate electrical signals representative of pressure transmitted to it through fluid located in the catheter lumen. Electrical signals representative of pressure can be communicated to processing circuitry 112 by way of an electrical pathway 108.

The optical unit is configured to transmit light into the subject and to receive light reflected by the subject. An optical unit can comprise a light source for transmitting the light and a photodetector to sample or detect received light. For example, the optical unit can be configured to transmit light into the blood vessel 102 lumen where the pressure is being measured.

The light can be transmitted from the optical unit into the subject through the lumen of the catheter 104 or through a fiber optic device of the catheter. Light reflected from the subject's blood is transmitted back to the optical unit through the catheter lumen or through the fiber optic device.

Returning light is detected or sampled and light signals representative of oxygen saturation are communicated to the processing circuitry 112 by way of the electrical pathway 108. Optionally, the processing circuitry can process received signals to provide measurements of blood pressure and oxygen saturation. Optionally, the processing circuitry 112 can process received signals and transmit data representative of oxygen saturation and pressure to a remote processing unit. The remote processing unit can process the received signals to provide measurements of blood pressure and oxygen saturation.

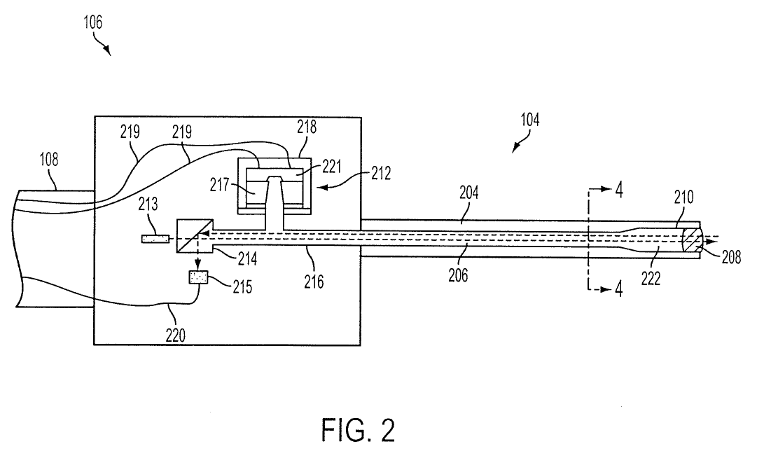

FIGS. 2-7 are schematic cross-sectional diagrams illustrating optional aspects of several embodiments of a device for sensing blood pressure and oxygen saturation such as the device 100 illustrated in FIG. 1. A device 100 comprises an sensor housing 106 and a pressure and light transmission catheter 104. Referring to FIGS. 2-7, the pressure and light transmission catheter comprises a wall portion 204 defining a lumen 206 that is in fluid communication with portions of the sensor housing 106. The pressure and light transmission catheter can be flexible or rigid. The pressure and light transmission catheter lumen 206 can be filled with a pressure transmitting fluid which communicates the pressure at the distal portion of the catheter to a proximal lumen end of the catheter. The pressure transmitting fluid can in some aspects also transmit light.

Thus, the lumen 206 can be filled with a fluid for transmitting pressure and light to the sensor housing 106. In this regard, the lumen of the pressure and light transmission catheter can be operatively placed in fluid communication with a pressure sensing unit 212 that is located within the sensor housing 106. The pressure sensing unit can comprise a pressure sensor.

The fluid can be any fluid that can transmit pressure and light along the length of the lumen 206. For example, a low-viscosity fluid can be used. The low-viscosity fluid in the lumen 206 can have minimal biological activity, can have a low thermal coefficient of expansion, can be insoluble in gel, can have a low specific gravity, can have a negligible rate of migration through the walls of the catheter, and can have a low viscosity at body temperature. As one example, the low-viscosity fluid is an inert perfluorocarbon.

The sensor housing 106 can further comprise an optical unit configured to generate light for transmission through the fluid of the lumen 206 and to receive light reflected from the subject's blood to determine oxygen saturation. Light reflected from the subject's blood is transmitted along the lumen 206 through the lumen fluid towards the optical unit, which is in operative communication with the lumen fluid. In this example, the optical unit is configured to direct light into the lumen and to receive light transmitted through the lumen fluid.

The optical unit can comprise a light source 213 and a photodetector 215. Optionally, the optical unit can further comprise a beam splitting device 214. Example light sources that can be used include, but are not limited to, a light emitting diode or laser. Optionally, the light source can generate red or infrared light. However, any suitable light source can be used that can deliver light to the subject's blood for measurement of oxygen saturation. Optionally, light from the light source is directed along a path through the beam splitter 214. After passing through the beam splitter, light can be directed into the lumen 206 of the pressure and light transmitting catheter. Light can also be directed into the lumen 206 without first passing through a beam splitter. If used, the beam splitter 214 can be a polarization sensitive beam splitter that transmits polarized light therethrough for direction into the lumen of the pressure and light transmission catheter.

Light travels through the pressure and light transmission catheter lumen 206 and exits the catheter lumen to interact with the subject's blood. A portion of the light transmitted into the subject's blood is reflected back into the lumen 206 of the pressure and light transmission catheter. Light reflected back into the lumen is directed to the photodetector 215. Optionally, the beam splitter 214 reflects at least a portion of the returning light to the photodetector 215 for detection. Light detected by the photodetector can be used to determine oxygen saturation. For example, an absorption extinction coefficient associated with the subject's blood can be determined based on the detected light, which can be used to measure oxygen saturation. Signals representative of detected pressure and light can be processed to determine blood pressure and oxygen saturation.

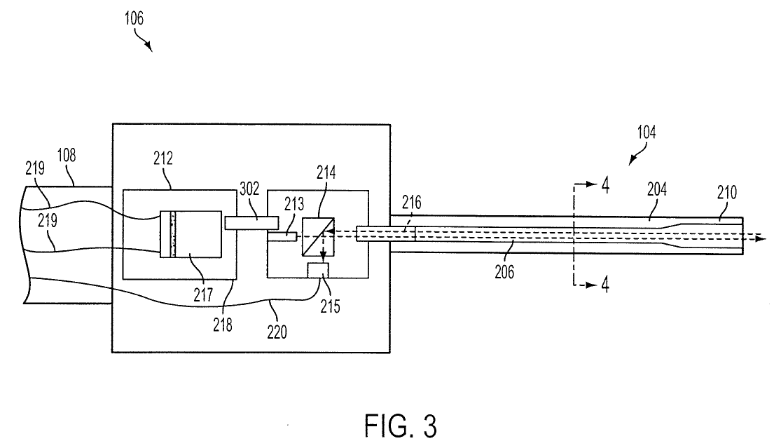

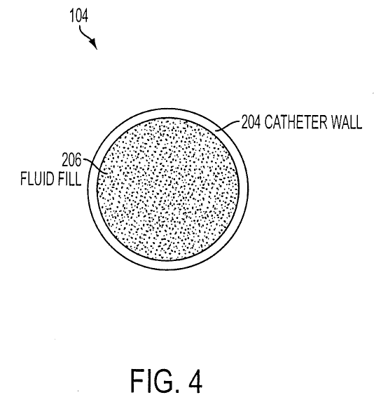

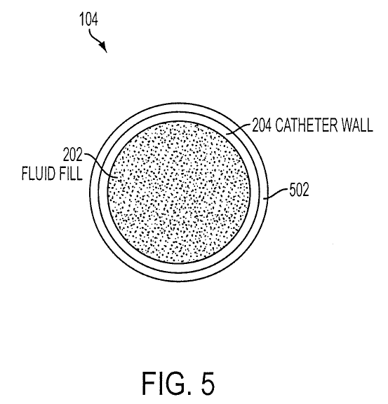

A schematic cross-section of the pressure and light transmission catheter across line 4-4 of FIG. 2 or FIG. 3 is shown in FIG. 4. FIG. 4 shows the catheter wall 204 and the lumen 206, which can be filled with fluid for transmitting light and pressure between the subject, the pressure sensing unit 212, and the optical unit. As shown in FIG. 5, optionally, the catheter wall 204 can be coated with a coating material 502. The coating 502 is configured to retain a higher percentage of light within the catheter lumen 206 as it is transmitted between the subject and the optical unit. The coating can also be located on the inside wall of the catheter lumen.

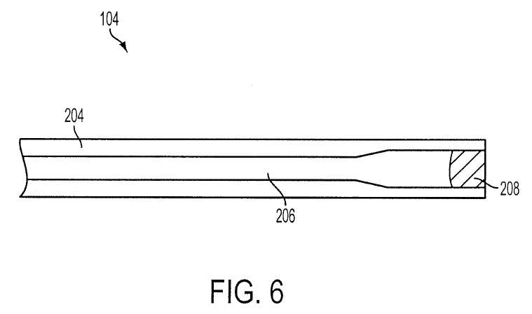

An example distal portion of the pressure and light transmission catheter 104 is shown by schematic cross-section in FIG. 6. The distal lumen portion of the pressure and light transmission catheter 104 can be positioned within the body of a human or animal at the site where pressure and/or oxygen saturation is to be measured. The pressure and light transmission catheter can be flexible or rigid. The pressure and light transmission catheter lumen 206 can be filled with a pressure transmitting fluid that can transmit light. Thus, a portion of pressure transmitting fluid that can transmit light at or about the distal portion interfaces with the body area where pressure and oxygen saturation is to be measured, such as with blood in an artery.

Optionally, the pressure and light transmission catheter can comprise a stem portion defining a lumen and a sheath fixed to the stem portion. The sheath comprises a wall that defines a cavity that is in fluid communication with the lumen. The pressure sensor is disposed in fluid communication with the lumen for transferring pressure between the cavity and a pressure transducer of the pressure sensing unit. In this aspect, the distal portion may be covered with a cap or plug. The cap or plug can allow the transmission of light, such that light can be transmitted from the optical unit through the lumen fluid and through the distal cap or plug. Similarly, reflected light can be received through the distal portion for transmission back to the optical unit. An example of a pressure transmitting catheter with a lumen, sheath and stem portion is described in United States Patent Publication Number 2008/0000303, “Pressure Sensing Device,” which is herein incorporated by reference. The pressure sensing catheter can be used as a pressure and light sensing catheter by directing light through its lumen to interrogate a subject's blood. For example, the catheter can be positioned in fluid communication with the electronics unit 106 similar to the pressure and light transmitting catheter shown in FIGS. 2 and 3. In this case, the distal tip, for example the distal plug, of the catheter can be modified to allow light transmitted along its lumen to pass therethrough for oxygen saturation detection and monitoring.

Referring again to FIGS. 2-6, the pressure sensing unit 212 comprises a pressure sensor. The pressure sensor can be adapted to transduce blood pressure into electrical signals. The pressure sensor can be a transducer 217 in communication with pressure transmitting fluid that can transmit light. Optionally, the transducer can be a solid state silicon piezoresistive bridge pressure sensor, which is mounted on a Pyrex pedestal, which in turn is mounted to a TO5 header and which is enclosed and hermetically sealed so as to operate in a sealed-gauge mode within metal can attached to TO5 header. The TO5 header comprises a hole centered on the pressure sensing unit to form a pressure access port in fluid communication with lumen 106 fluid. The operation and construction of a suitable pressure sensing unit including a transducer is described in detail U.S. Pat. No. 4,846,191, “Device for Chronic Measurement of Internal Body Pressure,” which is herein incorporated by reference.

Referring to FIG. 2, the transducer 217 can also comprise a cavity which is in fluid communication with the lumen 206 and in contact with the pressure and light transmitting fluid, so that pressure is transmitted through the fluid to a diaphragm 221 of the transducer 217. Optionally, movement of the diaphragm can be monitored using a light source and photodetector where movement of the diaphragm due to pressure within the blood vessel can be monitored by sampling light reflected from the diaphragm and received by the photodetector.

The transducer 217 responds to variations in the pressure transmitting fluid at the proximal end to provide an electrical pressure signal representing variations in the physiological pressure at the distal tip on electrical lead wires 219. Electrical lead wires 219 are carried within an electrical pathway 108. By separating the pressure and light transmission catheter 104 from the electrical pathway 108, the length of electrical pathway 108 can be independently determined from the length of the pressure and light transmission catheter.

An implantable device such as the device 100, can be a very small, lightweight device which can be implanted into animals as small as rats, to provide chronic measurements of blood pressure and oxygen saturation. A distal portion of the pressure and light transmission catheter 104 can be inserted into an artery of an animal to transmit the pressure of the fluid within the artery back to the pressure transducer 217 within the housing 218 of a pressure sensing unit 212. The sensed pressure is converted to electrical signals by circuitry, and a telemetry signal can be transmitted to a receiver external to the animal.

Electrical lead wires 219 and 220 can be coupled to the processing circuitry 112. The processing circuitry 112 can be powered by a battery. Telemetry can be employed to transmit signals representative of measurements of pressure and oxygen saturation to an external communications link that processes the signals for subsequent analysis. Thus, the processing circuitry can include a signal-processing and telemetry circuitry and a transmitting antenna for generating and transmitting a telemetry signal representing the pressure signal from the transducer and/or a signal from the photodetector 215 to an external receiver disposed outside of the human or animal.

The electrical pressure signal or oxygen saturation signal produced by the transducer or photodetector respectfully, can be amplified and filtered with signal-processing circuitry in the processing circuitry 112 and can also be modulated onto a radio-frequency carrier by the telemetry circuitry in an electronics module for transmission to the external receiver. Since the transducer 217 in this example can operate as a sealed gauge unit, atmospheric pressure can be subtracted from the measured pressure to provide gauge pressure. Measurement of atmospheric pressure can be obtained using an instrument designed for that purpose and subtraction can be performed by a remote computer system.

The catheter 104 can have a small diameter, hollow tube which is mounted at its proximal end over an intermediate conduit 216. The lumen 206 of catheter 104 can be filled with low viscosity liquid, which interfaces directly with the pressure sensor of the pressure sensing unit 212. Referring to FIG. 3, the pressure sensing unit 212 and optical unit are connected in fluid communication by a conduit 302.

Referring to FIG. 2 and FIG. 6, a membrane 208 can be optionally disposed at the distal tip of the pressure and light transmission catheter. A thin-walled section 210 can define an open cavity 222. As illustrated in FIG. 2, the membrane can be contained in a distal portion of open cavity 222. The open cavity is in fluid communication with the lumen 206. The portion of the open cavity 222 not filled with the membrane 208 and the lumen 206 can be filled with a low-viscosity fluid for transmitting light and pressure. The membrane can be, in one non-limiting example, a viscous gel membrane. In this way, physiological pressure can be transmitted from the distal tip of the pressure and light transmission catheter 104 through the walls of the pressure and light transmission catheter and via the membrane 208 contained within thin-walled section 222 to the low-viscosity fluid which communicates pressure directly to transducer of the pressure sensing unit 212. The low frequency components of the physiological pressure can be transmitted via the membrane 208 while the high frequency components of the physiological pressure can be transmitted through the walls of the pressure and light transmission catheter. The low-viscosity fluid can also function to transmit light from the light source to the subject and can transmit reflected light from the subject back to the optical unit for transmission to the photdetector.

Optionally, the membrane 208 is a hydrophobic gel material, which eliminates the possibility of osmotic pressure across the membrane 208 or migration of blood solutes into membrane 208. The length of the membrane 208 in cavity 222 of catheter 104 can be about one (1) millimeter to about three (3) millimeters. A loosely or minimally crosslinked silicone-based gel is one example of a material which provides biocompatibility and adequate frequency response.

The optical unit can also be used to monitor blood pressure. Blood pressure monitored by the optical unit can be used in addition to blood pressure measured by the pressure sensor 212. For example, the membrane 208 is positioned in the distal portion of the catheter and coupled to the fluid located in the catheter lumen. The membrane 208 can be a gel membrane as described above. A light source 213 is adapted for projecting light onto the membrane 208. A portion of the light passes through the membrane 208 to illuminate blood within the blood vessel lumen. A portion of the light is also modulated by the membrane 208 in response to blood pressure in the blood vessel lumen. The photodetector 213 is adapted to sample light reflected by the blood located in the blood vessel lumen and to sample light modulated by the membrane. The light modulated by the membrane 208 can be light that was transmitted onto and through the membrane and then reflected back through the membrane by the blood. The modulated light can represent blood pressure and the blood pressure can also be determined by the pressure sensor.

The catheter 104 can be manufactured from a biocompatible material with an optional outside diameter of about 0.5 to 1.5 millimeters and an optional inside diameter of about 0.3 to about 0.7 millimeters. The length of catheter 104 can depend on the particular animal involved. Optionally, the length is on the order of 5 to 6 centimeters for a rat and 15 to 25 centimeters for a dog. The inner diameter of cavity 222 of catheter 104 can be enlarged relative to the rest of catheter 104 in some applications, particularly when a very small catheter diameter is used. This reduces the distance the membrane 208 moves during thermal expansion and contraction of that portion of fluid located in pressure sensor. It also reduces movement of the membrane 208 due to changes in catheter internal volume induced by bending, thereby reducing artifact caused by flexing of catheter 104. In addition, the thin-walled portion 210 of catheter 104 can provide for an improved dynamic response due to the ability of the thin wall to transmit rapid changes in pressure from the blood into fluid contained within catheter lumen 206. Optionally, the pressure and light transmission catheter does not include an open cavity 222 defined by a thin-walled section 210, but instead, the small diameter portion of lumen 106 can extend all the way to the distal tip. The gel membrane can be disposed at the distal tip of the catheter with the remainder of lumen 206 being filled with the low-viscosity fluid.

The pressure and light transmission catheter 104 can be fabricated of a urethane material or other suitable biocompatible material. The viscous gel membrane, if used, can be a biocompatible and blood-compatible gel or other gel-like material that provides a direct interface with the tissue or fluid from which pressure is to be measured, such as blood in an artery. The membrane 208 can retain fluid within the lumen 206 and is of a viscosity higher than that of low viscosity fluid contained in the lumen. The viscous gel can comprise any material which is capable of flowing or moving within the pressure and light transmission catheter as does a viscous fluid or a plug that can slide or deform easily and contains intramolecular forces which make it very unlikely that any portion of this material will dissolve, break apart, slough off, or wash away when measuring physiological pressure within a human or animal. The membrane 208 can be viscous enough not to wash out of the catheter, but can also have a low enough viscosity that it can “flow” without significant pressure differential. For example, the viscous gel can be a silicone gel which contains cross-linked molecular entities. The distal tip of catheter 104 can be contoured to reduce trauma to the vessel and to inhibit turbulent flow when measuring blood pressure. The pressure and light transmission catheter 104 can be positioned in fluid communication with the pressure sensing unit 212 and the optical unit via a conduit 216.

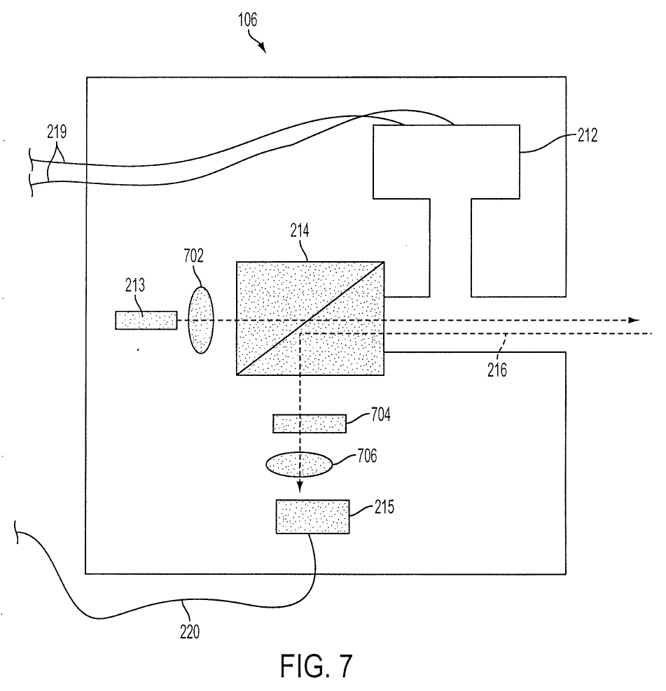

FIG. 7 is a schematic cross-sectional diagram showing optional aspects of the sensor housing 106 as shown in FIG. 1. The optical unit can comprise the light source 213, beam splitter 214 and photodetector 215. Optionally, the optical unit can further comprise a collimating lens 702 operatively positioned between the light source and the beam splitter in the path of light transmitted from the light source. Optionally, the optical unit can further comprise a filter 704 that selectively transmits the wavelength emitted by the light source 213. Optionally, the optical unit can further comprise a second lens 706 for focusing light onto the photodetector 215.

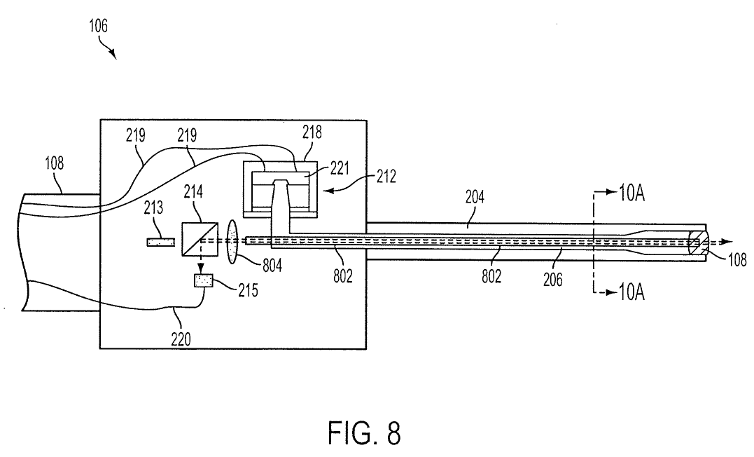

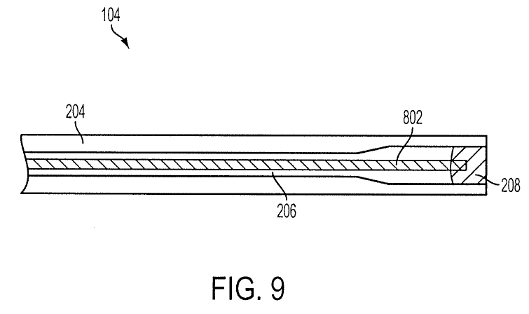

FIGS. 8-10 are schematic cross-sectional diagrams illustrating optional aspects of several additional embodiments of the device 100. A device 100 comprises a sensor housing 106 and a pressure and light transmission catheter 104. Referring to FIG. 8 and FIG. 9 the pressure and light transmission catheter 104 comprises a wall portion 204 defining a lumen 206 that is in fluid communication with portions of the sensor housing 106. The lumen 206 can be filled with a fluid for transmitting pressure to the sensor housing 106. In this regard, the lumen of the pressure and light transmission catheter can be operatively placed in fluid communication with a pressure sensor 212 that is located within the sensor housing 106. The sensor housing 106 can further comprise an optical unit configured to deliver light to a fiber optic device 802 of the pressure and light transmitting catheter for delivery to the subject. A portion of the light delivered to the subject is reflected and received by the fiber optic device 802 to determine or monitor the subject's oxygen saturation.

The optical unit can comprise a light source 213, a beam splitting device 214 and a photodetector 215. For example, the light source can be, but is not limited to, a light emitting diode or a laser. However, any suitable light source can be used that can deliver light to the subject's blood for measurement of oxygen saturation. Optionally, light from the light source is directed along a path through the beam splitter 214. Light can also be coupled to the fiber optic device 802. An example fiber optic device 802 is a single optical fiber. Multiple optical fibers can also be used to transmit and receive light. The beam splitter 214 can be a polarization sensitive beam splitter that transmits polarized light therethrough and into the fiber optic device 802. A lens 804 can be operatively positioned between the beam splitter 214 and fiber optic device to focus light into the fiber optic fiber or fibers for better coupling.

Light transmitted through the fiber optic device of the pressure and light transmitting catheter exits the fiber optic device to interact with the subject's blood. A portion of the light transmitted into the subject's blood is reflected back into the fiber optic device. Light reflected back into the fiber optic device, or a portion thereof, is transmitted to the photodetector 215. Separate fibers can be used for transmission of light into the subject's blood and for returning reflected light to the photodetector 215. Light detected by the photodetector can be used to determine oxygen saturation. For example, an absorption extinction coefficient associated with the subject's blood can be determined based on the returned light, which can be used to measure oxygen saturation. Optionally, the returning light can interact with the beam splitter 214, which can reflect at least a portion of the reflected light to the photodetector 215. Signals representative of detected pressure or light can be processed to determine blood pressure and oxygen saturation.

FIG. 9 shows a distal portion of a pressure and light transmission catheter. Optionally, the fiber optic device 802 can be positioned within the membrane 208 such that light transmitted from the fiber optic device and received by the fiberoptic device occurs through the membrane 208. Optionally, the fiber optic device 802 can terminate proximal to the membrane 208 such that it transmits and receives light through the full gel thickness. Moreover, the fiber optic device can also be optionally positioned so that transmitted and received light does not pass through the membrane 208.

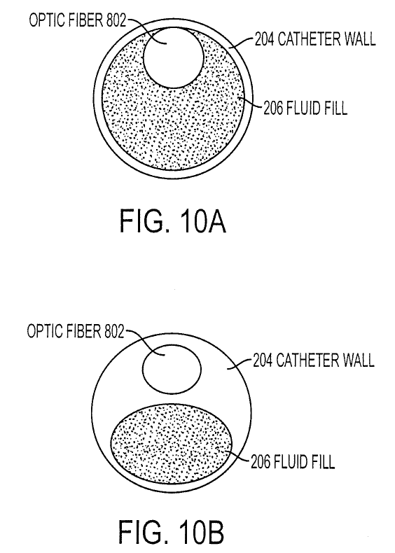

A schematic cross-section of an example pressure and light transmission catheter across line 10A-10A is shown in FIG. 10A. Another schematic cross-section of an example pressure and light transmission catheter is shown in FIG. 10B. FIG. 10A illustrates that an optical fiber of an example fiber optic device can be positioned within the lumen 206 of the catheter 104 to form the pressure and light transmission catheter. FIG. 10B illustrates that an optical fiber of a fiber optic device can be positioned within the catheter wall 204 to form the pressure and light transmission catheter. In another example, an optical fiber of a fiber optic device can be positioned in overlying registration with the outer catheter wall 104 to form the pressure and light transmission catheter.

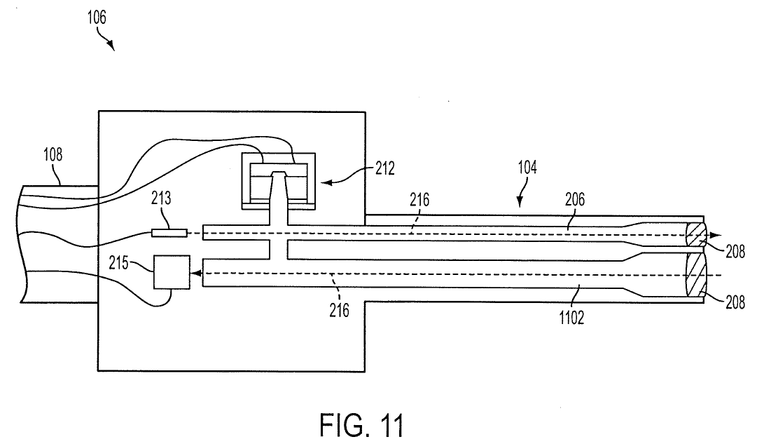

Referring to FIG. 11, the catheter 104 can have a second lumen 1102 in addition to the lumen 206. Both lumens can be filled with a pressure transmitting fluid that can also transmit light. In this embodiment, the optical unit can comprise a light source 213 positioned to project light into one of the two lumens. For example, the light can be projected into the lumen 206 for transmission into the cardiovascular system of the subject. Alternatively, light could be projected into the lumen 1102 for transmission into the cardiovascular system of the subject. Depending on which lumen has light projected into it by the light source 213, the other lumen can receive light energy reflected from blood in the subject's cardiovascular system and can transmit the received light to the light sensor or photodetector 215.

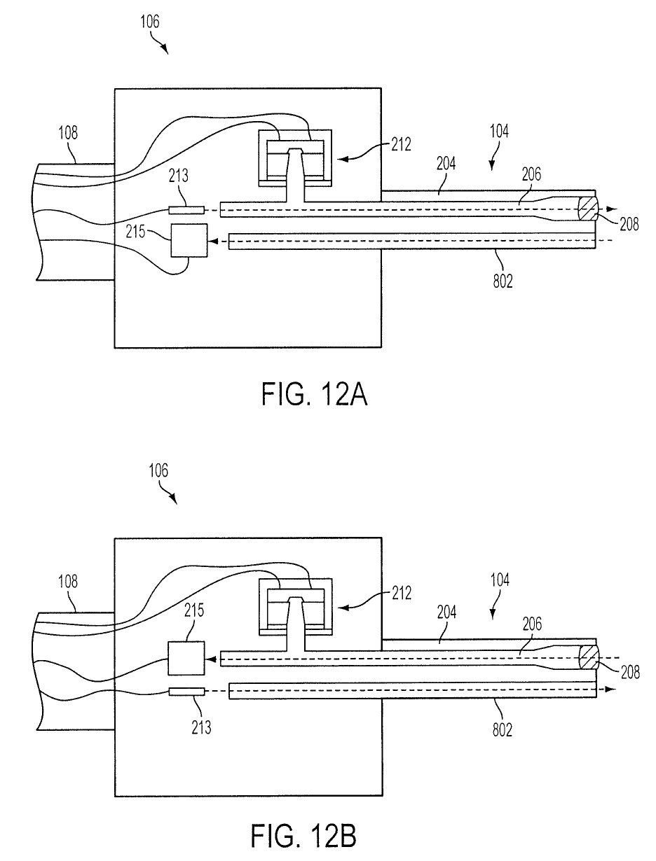

Referring to FIG. 12A and 12B, rather than two fluid filled lumens, the catheter can comprise one lumen 206 filled with fluid and a fiber optic device, such as a optical fiber 802. In FIG. 12A, the optical unit can comprise a light source 213 positioned to project light into the lumen 206, and the fiber optic device can receive light energy reflected from blood in the subject's cardiovascular system. The fiber optic device 802 can transmit the received light to the light sensor or photodetector 215. In FIG. 12B, the optical unit can comprise a light source 213 positioned to project light into the fiber optic device 802 for transmission into the subject's cardiovascular system and the lumen 206 can be positioned to receive light energy reflected from blood in the subject's cardiovascular system. The lumen 206 can transmit the received light to the light sensor 215.

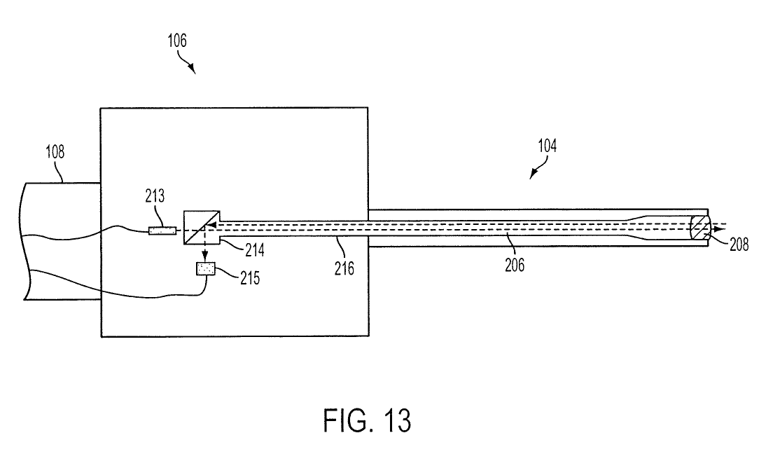

Referring to FIG. 13, a device for monitoring blood pressure and oxygen saturation comprises a fluid filled catheter 104 having a distal portion adapted for insertion within a blood vessel lumen of a living being. A membrane 208 is positioned in the distal portion of the catheter and coupled to the fluid located in the catheter lumen. The membrane 208 can be a gel membrane as described above. The membrane is adapted to be deflected by blood pressure in the lumen of the blood vessel.

A light source 213 is adapted for projecting light onto the membrane 208. A portion of the light passes through the membrane 208 to illuminate blood within the blood vessel lumen. The light source can also be adapted to project light into the blood using a fiber optic device. Light reflected by the subject's blood can return through the membrane for transmission to a photodetector 213. A portion of the light can be modulated by the membrane's deflection in response to blood pressure in the blood vessel lumen. The photodetector 213 is adapted to sample light reflected by the blood located in the blood vessel lumen and to sample light modulated by the membrane's deflection.

The light modulated by the membrane deflection can be light that was transmitted onto and through the membrane and then reflected back through the membrane by the blood. The light modulated by the membrane can also be light that was transmitted into the blood using a fiber optic device and reflected back onto the membrane. The photodetector can transduce or convert the sampled light into electrical signals. Optionally, signal processing circuitry can be electrically coupled to the photodetector for determining blood pressure and oxygen saturation measurements based upon the electrical signals transduced from the sampled light. Optionally, signal processing circuitry can be electrically coupled to the photodetector for remotely transmitting data for determining blood pressure and oxygen saturation based upon the electrical signals transduced from the sampled light.

The devices, systems, or portions thereof, can be implanted within a subject, including a human or non-human animal, for monitoring blood pressure and oxygen saturation. For example, in research animals, researchers have observed that when an animal's blood is sampled the oxygen saturation reading is generally very high and does not vary, which can indicate that a disturbed and excited animal may mask a low oxygen saturation reading. In another example, in poorly ventilated humans, excitement or talking can mask a low oxygen saturation. In both examples, because blood saturates quickly, oxygen saturation readings from blood samples can be artificially elevated due to excitement or sampling procedures. Blood pressure can also be elevated by excitement or sampling procedures. The described devices and systems may be used to monitor blood pressure and oxygen saturation and can reduce unwanted elevated oxygen saturation readings. The devices may be used to monitor research animals to provide oxygen saturation readings less affected by temporary excitement induced by handling, while also providing accurate blood pressure readings. The devices may also be used clinically in human or veterinary patients. For example, patients where oxygen saturation is being chronically measured, ventilated patients, or those with heart failure or receiving oxygen therapy may be advantageously monitored using the described devices.

A number of embodiments of the invention have been described. Nevertheless, it will be understood that various modifications may be made without departing from the spirit and scope of the invention. Accordingly, other embodiments are within the scope of the following claims.

What is claimed is:

1. An implantable device for monitoring blood pressure and oxygen saturation in a living being, comprising:
a fluid filled catheter having a distal portion adapted for insertion within a blood vessel lumen of the living being;
a pressure sensor in communication with the fluid, the fluid communicating blood pressure to the pressure sensor for transduction of the blood pressure into electric signals;
a light source adapted for illuminating blood within the blood vessel lumen; and
a photodetector adapted to sample light reflected by the blood located in the blood vessel lumen and that transduces the sampled light into electrical signals.

2. The implantable device of claim 1, further comprising signal processing circuitry electrically coupled to the pressure sensor and photodetector for determining blood pressure and oxygen saturation measurements based upon the electrical signals transduced from the blood pressure and the electrical signals transduced from the sampled light.

3. The implantable device of claim 1, further comprising signal processing circuitry electrically coupled to the pressure sensor and photodetector for remotely transmitting data for determining blood pressure and oxygen saturation based upon the electrical signals transduced from the blood pressure and the electrical signals transduced from the sampled light.

4. The implantable device of claim 1, wherein the light source is positioned in relation to the catheter to project light into the fluid of the catheter for illuminating the blood within the vessel.

5. The implantable device of claim 4, wherein the fluid is configured to transmit the light projected into it.

6. The implantable device of claim 5, wherein the light projected into the fluid is directed into the blood vessel lumen by transmission through the fluid.

7. The implantable device of claim 6, wherein the photodetector is positioned in relation to the catheter to sample the light reflected by blood located in the vessel lumen.

8. The implantable device of claim 7, wherein the light reflected by the blood located in the vessel lumen is guided to the photodetector for sampling by transmission into and through the fluid.

9. The implantable device of claim 1, further comprising an optical fiber, wherein the light source is positioned to project light into the optical fiber, the optical fiber being adapted to transmit the light received from the light source to illuminate blood within the blood vessel.

10. The implantable device of claim 9, wherein the optical fiber is adapted to receive light reflected by blood located within the blood vessel lumen and to transmit the received reflected light to the photodetector for sampling.

11. The implantable device of claim 10, wherein the photodetector is positioned in relation to the optical fiber to sample the reflected light transmitted to it by the optical fiber.

12. The implantable device of claim 9, further comprising a second optical fiber adapted to receive light reflected by blood within the blood vessel lumen and to transmit the received reflected light to the photodetector for sampling.

13. The implantable device of claim 1, wherein the catheter has at least a first and second lumen each being fluid filled.

14. The implantable device of claim 13, wherein the light source is positioned to project light into the first fluid filled lumen for illuminating blood with the blood vessel lumen and wherein the photodetector is positioned to sample light reflected by blood within the blood vessel lumen and transmitted to the photodetector through the fluid of the second lumen.

15. The implantable device of claim 1, further comprising an optical fiber positioned to receive light reflected by blood located in the blood vessel lumen, wherein the optical fiber is adapted to transmit the received reflected light to the photodetector for sampling.

16. The implantable device of claim 15, wherein the light source is positioned to project light into the fluid for illuminating blood within the blood vessel lumen and the photodetector is positioned to sample light reflected by the blood and transmitted to the photodetector through the optical fiber.

17. The implantable device of claim 9, wherein the light source is positioned to project light into the optical fiber and wherein the photodetector is positioned to sample light reflected by blood within the blood vessel lumen and transmitted to the photodetector through the fluid.

18. A system for monitoring blood pressure and oxygen saturation in a living being, comprising:
an implantable device, wherein the implantable device comprises:
a fluid filled catheter having a distal portion adapted for insertion within a blood vessel lumen of the living being;
a pressure sensor in communication with the fluid, the fluid communicating blood pressure to the pressure sensor for transduction of the blood pressure into electric signals;
a light source adapted for illuminating blood within the blood vessel lumen;
a photodetector adapted to sample light reflected by the blood located in the blood vessel lumen and that transduces the sampled light into electrical signals; and
signal processing circuitry electrically coupled to the pressure sensor and photodetector for remotely transmitting data for determining blood pressure and oxygen saturation based upon the electrical signals transduced from the blood pressure and the electrical signals transduced from the sampled light; and
wherein the system further comprises a remote processing unit located external to the living being and configured to receive the transmitted data and to determine blood pressure and oxygen saturation values from the received transmitted data.

19. The system of claim 18, wherein the light source is positioned in relation to the catheter to project light into the fluid of the catheter for illuminating the blood within the vessel.

20. The system of claim 19, wherein the fluid is configured to transmit the light projected into it.

21. The system of claim 20, wherein the light projected into the fluid is directed into the blood vessel lumen by transmission through the fluid.

22. The system of claim 21, wherein the photodetector is positioned in relation to the catheter to sample the light reflected by blood located in the vessel lumen.

23. The system of claim 22, wherein the light reflected by the blood located in the blood vessel lumen is guided to the photodetector for sampling by transmission into and through the fluid.

24. The system of claim 18, further comprising an optical fiber, wherein the light source is positioned to project light into the optical fiber, the optical fiber being adapted to transmit the light received from the light source to illuminate blood within the blood vessel.

25. The system of claim 24, wherein the optical fiber is adapted to receive light reflected by blood located within the blood vessel lumen and to transmit the received reflected light to the photodetector for sampling.

26. The system of claim 25, wherein the photodetector is positioned in relation to the optical fiber to sample the reflected light transmitted to it by the optical fiber.

27. The system of claim 24, further comprising a second optical fiber adapted to receive light reflected by blood within the blood vessel lumen and to transmit the received reflected light to the photodetector for sampling.

28. The system of claim 18, wherein the catheter has at least a first and second lumen each being fluid filled.

29. The system of claim 28, wherein the light source is positioned to project light into the first fluid filled lumen for illuminating blood with the blood vessel lumen and wherein the photodetector is positioned to sample light reflected by blood within the blood vessel lumen and transmitted to the photodetector through the fluid of the second lumen.

30. The system of claim 18, further comprising an optical fiber positioned to receive light reflected by blood located in the blood vessel lumen, wherein the optical fiber is adapted to transmit the received reflected light to the photodetector for sampling.

31. The system of claim 30, wherein the light source is positioned to project light into the fluid for illuminating blood within the blood vessel lumen and the photodetector is positioned to sample light reflected by the blood and transmitted to the photodetector through the optical fiber.

32. The system of claim 24, wherein the light source is positioned to project light into the optical fiber and wherein the photodetector is positioned to sample light reflected by blood within the blood vessel lumen and transmitted to the photodetector through the fluid.

33. An implantable device for monitoring blood pressure and oxygen saturation in a living being, comprising:
a fluid filled catheter having a distal portion adapted for insertion within a blood vessel lumen of the living being;
a membrane positioned in the distal portion of the catheter and coupled to the fluid, the membrane adapted to be deflected in response to blood pressure in the blood vessel lumen;
a light source adapted for illuminating blood with the blood vessel lumen; and
a photodetector adapted to sample light reflected by the blood located in the blood vessel lumen and to sample light modulated by deflection of the membrane, wherein the photodetector transduces the sampled light into electrical signals.

34. The implantable device of claim 33, further comprising signal processing circuitry electrically coupled to the photodetector for determining blood pressure and oxygen saturation measurements based upon the electrical signals transduced from the sampled light.

35. The implantable device of claim 33, further comprising signal processing circuitry electrically coupled to the photodetector for remotely transmitting data for determining blood pressure and oxygen saturation based upon the electrical signals transduced from the sampled light.
